(19) United States
(12) Patent Application Publication
(10) Pub. No.: US 2005/0216204 A1
(43) Pub. Date: Sep. 29, 2005

(54) Method and apparatus for tissue modeling

(76) Inventors: Bulent Yener, Canaan, NY (US); S. Humayun Gultekin, Portland, OR (US); Cigdem Gunduz, Troy, NY (US)

Correspondence Address: Schmeiser, Olsen & Watts LLP, Suite 201, 3 Lear Jet Lane, Latham, NY, 12110, US

(21) Appl. No.: 11/082,412

(22) Filed: Mar. 17, 2005

Related U.S. Application Data
(60) Provisional application No. 60/554,107, filed on Mar. 18, 2004.

(51) Int. Cl. G06F 19/00; G01N 33/48; G01N 33/50

(52) U.S. Cl. 702/19

(57) ABSTRACT

A method and apparatus for tissue modeling using at least one tissue image derived from clinical tissue. The at least one tissue image having cells therein. The method comprises for each tissue image of the at least one tissue image wherein each tissue image is denoted as a sample tissue image: clustering data derived from the sample tissue image to generate cluster vectors, each cluster vector representing of portion of the tissue image; generating cell information, comprising assigning a cell class or a background class to each of the cluster vectors; generating a cell-graph for the sample tissue image from using the generated cell information, said cell-graph comprising nodes and edges, said edges connecting some of the cell nodes together based on a connectivity criterion; and computing at least one metric from the generated cell-graph.

### RELATED APPLICATION

The present invention claims priority to U.S. Provisional Application No. 60/554,107, filed Mar. 18, 2004 and entitled “Cell-graphs: a method and apparatus for cancer modeling for noninvasive diagnosis”, and is incorporated herein by reference in its entirety.

### BACKGROUND OF THE INVENTION

1. Technical Field

The present invention relates to a method and apparatus for tissue modeling using at least one tissue image derived from clinical tissue that has been surgically removed from at least one patient.

2. Related Art

Cancer is an uncontrolled proliferation of cells that express varying degrees of fidelity to their precursors. Neoplastic process entails not only cellular proliferation but also a modification of the differentiation of the involved cell types. Thus, in a sense cancer may be viewed as a burlesque of normal development. See E. Rubin and J. L. Farber, Pathology, 2nd Ed., Lippincott, PA 1994.

Diffuse malignant gliomas are cancerous brain tumors that invade the surrounding normal tissue by an aggressive diffusion process. This diffuse invasive behavior affects the prognosis adversely, and renders radical treatment impossible. Current mathematical models to quantify and analyze a cancer tumor are not scalable due to their enormous complexity.

Such diffuse gliomas possess the capability to infiltrate the surrounding healthy brain tissues by an initially non-destructive migrational manner. The biological basis for glioma invasion constitutes a complex process involving cell-to-cell interaction, adhesion to the exctracellular matrix, tumor cell motility, and enzymatic remodeling of the extracellular space. See P. Lantos, D. N. Louis, M. K. Rosenblum, P. Kleihuis, “Tumors of the Nervous System”, in Greenfield's Neuropathology, 7th Ed. Vol. 2 pp 767-1052 Eds: D. Graham & P. Lantos, Oxford University Press, London 2002. Although the state of art medical imaging improved the detection of gliomas; quantification of the extent of invasion, prediction of biological behavior, and radical surgical removal in individual cases remains a challenge.

Mathematical modeling of cancer and quantification of its properties has been a focus of intensive research. See Cancer Modeling ed: J. Thompson and B. Brown, Marcel Dekker, Inc. 1987. See also M. A. J. Chaplain, “The Mathematical Modelling of Tumor Angiogenesis and Invasion”. Acta Bzotheoret., 43:387-402, 1995. See also D. Drasdo, R. Kree and J. S. McCaskill, “Monte-Carlo Approach to Tissue Cell Populations”, Phys. Rev E, 52(6B):6635-6657, 1995. See also A. Anderson, M. Chaplain, E. Newman, R. Steele and A. Thompson, “Mathematical Modelling of Tumor Invasion and Metastasis”, J. Theor. Med. 2:129-165,2000. See also S. Turner and J. Sherratt, “Intercellular Adhesion and Cancer Invasion: A Discrete Simulation Using the Extended Potts model”, J. Theor. Biol., 216:85-100, 2002.

However, current computational and mathematical models at the cellular level are not scalable. Some of these approaches are based on Monte-Carlo algorithm. See D. Drasdo, R. Kree and J. S. McCaskill, “Monte-Carlo Approach to Tissue Cell Populations”, Phys. Rev E, 52(6B):6635-6657, 1995. See also S. Turner and J. Sherratt, “Intercellular Adhesion and Cancer Invasion: A Discrete Simulation Using the Extended Potts model”, J. Theor. Biol., 216:85-100, 2002.

Other computational and mathematical models are based on formulating continuous differential equations and finding probability generating functions to model the cell behavior. Clearly, solving large number of equations or simulating millions or billions of cells with Monte-Carlo algorithms has prohibitive computational complexity. Thus, addressing the scalability problem requires new algorithmic approaches and new models.

### SUMMARY OF THE INVENTION

The present invention provides a method for tissue modeling using at least one tissue image derived from clinical tissue, said at least one tissue image having cells therein, said method comprising for each tissue image of the at least one tissue image wherein each tissue image is denoted as a sample tissue image:

clustering data derived from the sample tissue image to generate cluster vectors, each cluster vector representing of portion of the tissue image;

generating cell information, comprising assigning a cell class or a background class to each of the cluster vectors;

generating a cell-graph for the sample tissue image from using the generated cell information, said cell-graph comprising nodes and edges, said edges connecting some of the cell nodes together based on a connectivity criterion; and

computing at least one metric from the generated cell-graph.

The present invention provides an apparatus for implementing the aforementioned method, said apparatus comprising:

means for clustering the data derived from the sample tissue image;

means for generating the cell information;

means for generating the cell-graph for the sample tissue image; and

means for computing the at least one metric.

The present invention advantageously provides a method using a graph theoretical model that is scalable.

### BRIEF DESCRIPTION OF THE DRAWINGS

FIG. 1 is a flow chart depicting a method for cancer modeling for noninvasive diagnosis, in accordance with embodiments of the present invention.

FIG. 2 depicts a single perceptron, in accordance with embodiments of the present invention.

FIG. 3 depicts a multilayer network comprising perceptrons, in accordance with embodiments of the present invention.

FIGS. 4-5 depict images representing a methodology for graphically representing cells of biological tissue, in accordance with embodiments of the present invention.

FIG. 6 depicts cell-graphs representing cancer and normal cells, in accordance with embodiments of the present invention.

FIG. 7 depicts data histograms of metrics computed for the cell-graphs representing cancer and normal cells in FIG. 6, in accordance with embodiments of the present invention.

FIG. 8 depicts images and cell-graphs representing cancer and inflammation cells, in accordance with embodiments of the present invention.

FIG. 9 depicts data histograms of metrics computed for the image and cell-graphs representing cancer and inflammation cells in FIG. 8, in accordance with embodiments of the present invention.

FIG. 10 depicts data histograms of metrics computed for the cell-graphs representing cancer cells and for randomly generated cell-graphs, in accordance with embodiments of the present invention.

FIG. 11 depicts an image and graph of tissue containing both cancer and normal cells and a graph classifying cancer and normal cells within the image, in accordance with embodiments of the present invention.

FIG. 12 depicts image processing of cancerous tissue showing a cancerous glioma tissue image, in accordance with embodiments of the present invention.

FIG. 13 illustrates a comparison between normal tissue and cancer tissue, in accordance with embodiments of the present invention.

### DETAILED DESCRIPTION OF THE INVENTION

The detailed description of the present invention is organized into the following sections: Introduction; Formalism and Methodology; and Experiments.

Introduction

The present invention provides novel mathematical techniques to model a cancer tumor and to quantify the properties of the invasion of biological tissue by cancer cells. The present invention uses a macroscopic modeling rather than cellular modeling in which tissue is represented by graphs and each node can represent a bunch of cells instead of a single cell.

A machine learning algorithm of the present invention uses a scalable, graph theoretical model, based on examination of the coordinates of individual cells in a sample tissue to construct a cell-graph for determining a spatial relationship between the cells of biological tissue. The mathematical properties of the cell-graph are computed by the machine learning algorithm to identify subgraphs that represent different biomedical phenomena in the sample tissue. The machine learning algorithm is trained over numerous samples under human (expert) supervision. The machine learning algorithm uses graph metrics to distinguish: (i) gliomas from surrounding normal tissue; and (ii) gliomas from other invasions such as inflammation. The machine learning algorithm has been tested, using real data derived from tissue samples, to validate the methodology of the present invention.

The graph theoretical approach of the present invention is motivated by the fact that many real-world, self-organizing, complex dynamic systems can be represented by graphs. Furthermore, precise metrics are available to quantify the properties of these graphs in such systems and identify their characteristics. One example is the Hollywood movie star network, obtained by drawing a line between two actors if they played in the same movie. This network is derived from 150,000 movies and has 300,000 nodes. Another example is the World Wide Web (WWW) graph in which each page is a node and each Universal Resource Locator (URL) is a directed link. This WWW graph has billions of nodes and several billions of links (it was based on 1999 data). Similarly, the Internet router graph has hundreds of thousands nodes and links. Another example is the USA power grid network which has approximately 5,000 nodes. A collaboration network among the mathematicians with 70,000 nodes and 200,000 links (1991-1998 data) is another example. In addition, the tiny neural network of C-elegance worm with 300 nodes (neurons) shares common properties with the earlier mentioned, much large networks. Although the size and domains of these graphs are very different, it is possible to distinguish them from random graphs (see B. Bollabas, Random Graphs (Academic Press, London, 1985)) using some of the metrics that are adapted in this work as well.

The approach of the present invention is based on construction of cell-graphs from the tissue images. A cell-graph is denoted by G=(V, E) where the vertex (node) set represents the nucleus of cells and the edge set E defines a locality relationship between the nodes.

The results described infra herein demonstrate that a cell-graph derived from sample tissue images and deployment of a machine learning algorithm distinguishes between different regions in the tissue based on the graph metrics. The graph theoretical model of the present invention is scalable, since graphs with order of millions nodes can be tackled to compute the metrics of interest.

Formalism and Methodology

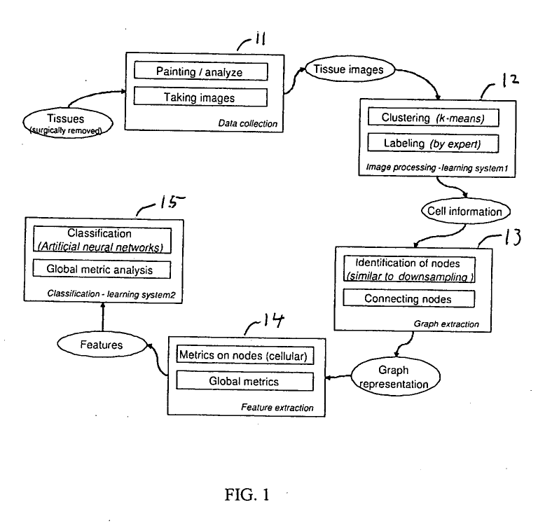

FIG. 1 is a flow chart depicting a method for cancer modeling for noninvasive diagnosis, in accordance with embodiments of the present invention. The flow chart comprises steps 11-15.

Step 11 (“Data collection”) obtains tissue images derived from surgically removed clinical tissue from patients. A staining process enables the tissue images to be seen under a microscope. Using these images of tissue samples, the inventive tool of steps 12-15 distinguishes and recognize different type of cells; e.g., healthy, cancer, or inflamed cells.

Step 12 (Image processing-learning systems”) determine the cell locations in a tissue image by distinguishing the cells from their background. A K-means clustering algorithm, based on the color information of the pixels (see J. A. Hartigan and M. A. Wong, “A K-Means Clustering Algorithm”, Applied Statistics, vol. 28, pp. 100-108,1979; Advances in Physics, cond-mat/0106144, 2002), is used. After setting the cluster vectors on training samples, a pathology expert analyzes the cluster information and assigns classes to the cluster vectors; i.e., the pathology expert labels these clusters as one (1) for cell regions, or as zero (0) for background (i.e., non-cell) regions. These labeled clusters are used in the tissue samples during testing.

The K-means clustering algorithm is an unsupervised learning algorithm that clusters the data based on their features. See J. A. Hartigan and M. A. Wong, “A K-Means Clustering Algorithm”, Applied Statistics, vol. 28, pp. 100-108,1979; Advances in Physics, cond-mat/0106144, 2002. The K-means algorithm is applied to K cluster vectors and each sample belongs to one of the clusters whose center is the closest to that sample. After assigning the sample to one of the clusters, the sample is represented by this cluster vector.

The K-means algorithm is trained as to minimize the distances between the samples and their corresponding cluster vectors. Beginning with random cluster vectors, and after assigning each sample to its closest vector, cluster vectors are recomputed as the mean of all samples that belong to them. This continues iteratively until reaching a convergence point.

The K-means algorithm is used to cluster the color information of the tissue images, where the color information is represented by red-green-blue (RGB) values. Each cluster vector, which is also composed of RGB values, represents the group of colors.

The K-means algorithm is unsupervised learning and after learning, these clusters are labeled (e.g., by a pathology expert as stated supra) as one (1) for cell regions or as zero (0) for background (i.e., non-cell) regions as stated supra.

Step 13 (“Graph extraction”) transforms the cell information to identify the nodes (vertices) of the graph. A potential difficulty is noise, since in glioma samples there are too many cells with different sizes as well as coinciding cells. The noise prevents a one-to-one mapping between a cell and a node. Moreover, if a one-to-one mapping were possible, then the number of nodes in the graph would be dependent on the number of cells, which makes the computation hard for very large tissue cells.

The present invention approaches the aforementioned problem by having the transformation of the cell information in step 13 embed a two-dimensional grid over the sample image and calculate the probability of a grid entry being a cell. For each grid entry, the probability value is computed as the average of the label of pixels located in this entry. A threshold (i.e., node-threshold) is applied to the computed probability values and the values greater than the node-threshold are labeled as cells, whereas the others are labeled as background. The labeling of cells and background is governed by two control parameters, namely: (i) the size of the grid (e.g., number of nodes); and (ii) the node-threshold value.

Use of the two-dimensional grid may be considered as a downsampling of the image obtained in step 12. Increasing the node-threshold value produces sparser graphs, and the grid size determines the downsampling rate. Note that the resolution of a tissue image determines the complexity of whole process.

Thus, the labeling of the grid entries as cell or background translates the spatial information of the nodes to their locations on the two-dimensional grid. After the nodes are translated to their locations on the two-dimensional grid, edges are defined to connect the nodes to construct the graph. Defining the edges uses the locations of the nodes in the two-dimensional grid. Any two nodes are to be connected by an edge if the distance between the two nodes is smaller than a predefined edge-threshold. Thus, the edge threshold affects the connectivity of the graph. Increasing the edge-threshold results in denser graphs

Step 14 (“Feature extraction”) computes six different metrics on the resultant graphs, reflecting the different topological properties of the graphs and providing information of its characteristics. The metrics defined herein may be used in analyzing the other types of graphs, e.g., Internet, actor or C-elegance worm graphs. These metrics quantify the information about the degree distribution of a node, the connectivity information of its neighbors, and the connectedness information of itself as well as the whole graph. Metrics defined on the nodes are local, but by using statistics, the metrics also provide the global information for the graph. A precise mapping from these metrics to properties of glioma cells is outside the scope of the description herein. The six metrics are used herein to identify and distinguish mathematical properties of gliomas from other cell structures. The six metrics are: degree, clustering coefficient Cᵢ, clustering coefficient Dᵢ, closeness, betweenness, and eccentricity.

The “degree” metric is defined as the number of the connections of a single node for an undirected graph. Its value on a tumor graph is higher, but the higher degree values are not always an indicator of a cancer.

A clustering coefficients reflects the connectivity information in the neighborhood environment of a node. See S. N. Dorogovtsev and J. F. F. iilendes, “Evolution of Networks”, Advances in Physics, cond-mat/0106144, 2002. The clustering coefficients provide the transitivity information (see M. E. J. Newman, “Who is the Best Connected Scientist? A Study of Scientific Coauthorship Networks”, Phys. Rev., cond-mat/O011144, 2001), since a clustering coefficient controls whether two different nodes are connected or not, if they are connected to the same node. The present invention utilizes clustering coefficients Cᵢ and Dᵢ.

The clustering coefficient Cᵢ is defined as the percentage of the connections between the neighbors of node i, and is given as
Cᵢ=2Eᵢ/(k·(k−1)) (1)
where k is the number of neighbors of node i, and Eᵢ is the existing connections between its neighbors.

Random and scale-free graphs can be distinguished by using the clustering coefficient C. Random graphs have small values of clustering coefficients C, whereas scale-free graphs have larger values than those of the random graphs. The inventors of the present invention have observed larger values for their tissue images, which indicates the scale-free-ness of the graphs and also demonstrates that the cell-graphs are not random.

The clustering coefficient Dᵢ is a modified version of the clustering coefficient defined in S. N. Dorogovtsev and J. F. F. iilendes, “Evolution of Networks”, Advances in Physics, cond-mat/0106144, 2002. Clustering coefficient Dᵢ, which is similar to Cᵢ with an exception of taking into account node i and its connections, is given as:
Dᵢ=2·(Eᵢ+k)/(k·(k+1)) (2)

“Closeness” and “betweenness” are local metrics that measure the connectedness of a graph. See M. E. J. Newman, “Who is the Best Connected Scientist? A Study of Scientific Coauthorship Networks”, Phys. Rev., cond-mat/O011144, 2001. The closeness of a node is the average of the distances between the node and every other nodes except itself. Closeness reflects the centrality property of a single node and smaller values indicate that this node places close to the center of a graph. Betweenness of a node is the total number of the shortest paths that pass through the node. These metrics may indicate the location of a cell within the tumor. For example, having a smaller closeness value or higher betweenness value may suggest that the cell is close to the center of the tumor.

“Eccentricity” of a node is a local metric defined as the minimum number of hops required to reach at least 90 percent of its reachable nodes. The higher values of this metric may indicate the density of the diffuse invasion.

Step 15 of FIG. 1 (“Classification”) executes a machine learning algorithm, using the metrics computed in step 14 as input, to classify different cell concentrations as cancerous, normal, or inflammation. The machine learning algorithm employs artifical neural networks.

A neural network comprises nodes, called “perceptrons”, that are tied with weighted connections. Each perceptron takes a vector of input values and computes a single output value as the weighted sum of its input values. The output value is activated only if the output value exceeds the threshold defined by an activation function. See C. M. Bishop, Neural Networks for Pattern Recognition, Oxford University Press, 1995. See also A. K. Jain, J. Mao and K. M. Mohiuddin, “Artificial Neural Networks: A Tutorial”, Computer, Vol. 29, No. 3, pp. 31-44, 1996.

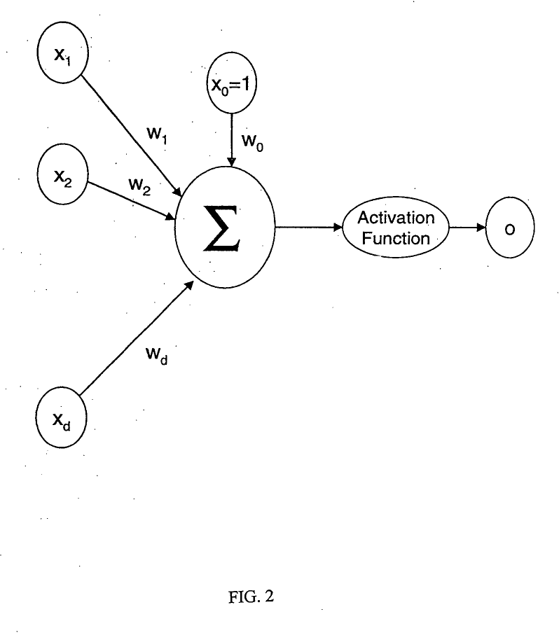

FIG. 2 depicts a single perceptron inputs xᵢ and output (o), in accordance with embodiments of the present invention. Weights wᵢ are associated with each input xᵢ, where wₒ is a bias term. The present invention uses multilayer perceptrons. The outputs of each layer are connected to the inputs of another layer. The inputs, xᵢ are the topological metrics and the output (o) is the class label, indicating whether a cell is cancerous, healthy, or generated as synthetically. The input layer is connected to a hidden layer with weights wᵢⱼ and the hidden layer connects to an output layer with weights vᵢⱼ.

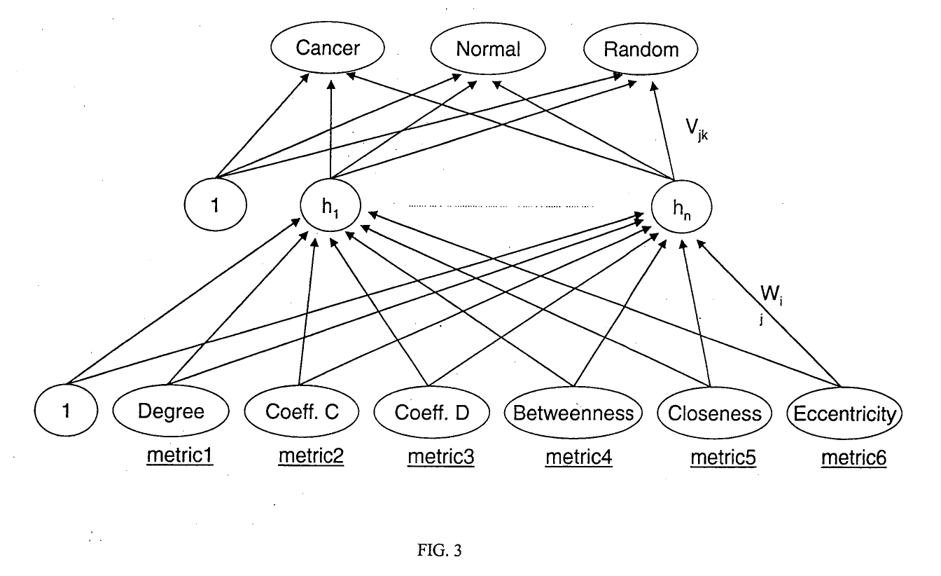

FIG. 3 depicts a multilayer network comprising perceptrons, in accordance with embodiments of the present invention. The inputs are the local metrics defined for the nodes of the extracted graphs. The output indicates whether a cell is cancerous, healthy, or generated synthetically. The outputted cell classification makes use of the six different local metrics, described supra.

Experiments

Experiments were conducted on clinical data for brain tumors, wherein the digital images of surgically removed tissues were used to construct a graph representing the data as explained supra. Each pixel of these images is represented by its RGB values.

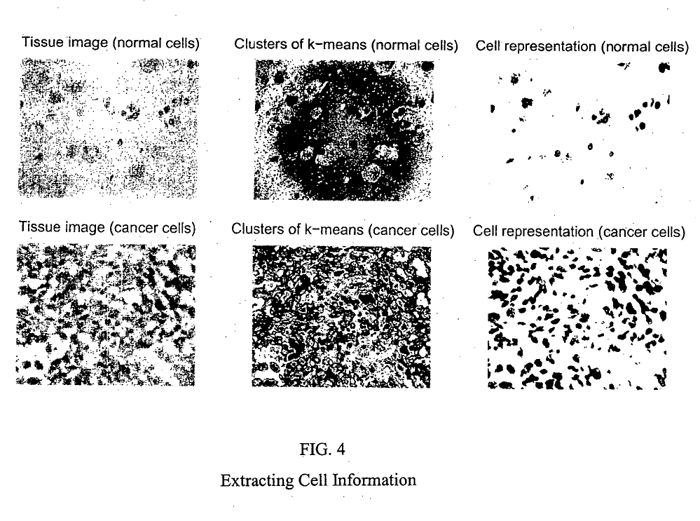

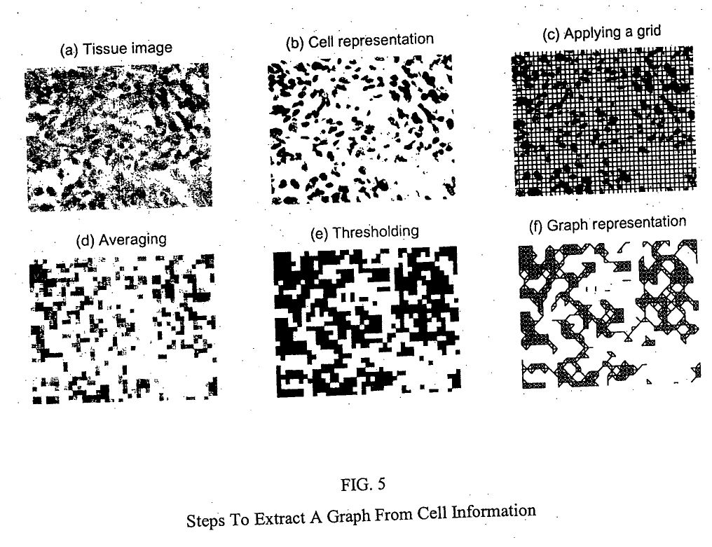

FIGS. 4-5 depict images representing a methodology for graphically representing cells of surgically removed tissue, in accordance with embodiments of the present invention.

FIG. 4 illustrates step 12 of FIG. 1 in which cell information is extracted from the surgically removed tissue. The K-means algorithm (described supra) was run on the data to learn cluster vectors on training samples. These cluster values are used for the test samples. Various K values were tried, and based on the clusters and based on human expertise, the clusters were labeled as either cell or background. FIG. 4 illustrates these steps for both cancer and normal tissues. The images in this graph in FIG. 4 are from the test set and are not used in training. The value of K is selected as 17 in this graph in FIG. 4.

After determining the cell and background regions as discussed supra in conjunction with FIG. 4, the nodes are to be extracted on these data, as illustrated in FIG. 5 in relation to step 13 of FIG. 1. A tissue image having cancer cells therein and the tissue's cell representation are depicted in FIGS. 5(a) and 5(b), respectively. In FIG. 5(c), a grid has been embedded on the cell representation of FIG. 5(b). For each entry of a grid of FIG. 1(c), a probability value of having a cell is computed by averaging the labeled data in the grid entry. FIG. 5(d) uses gray scale levels to represent the average values. Note that cell regions are labeled as 1 and the background is labeled as 0. A pair of cells of FIG. 5(d) are connected if the distance between them is smaller than a edge-threshold, as shown in FIG. 5(e). These three parameters are set as follows: the grid size=50 (i.e., 50 pixels are grouped to represent a cell or not); the node-threshold=0.1 (i.e., at least 10 percent of a grid entry should consist of cell regions to being a cell); and the edge-threshold=1 (i.e., two nodes are to be connected if they are adjacent in the grid. The resultant graph representation is shown in FIG. 5(f).

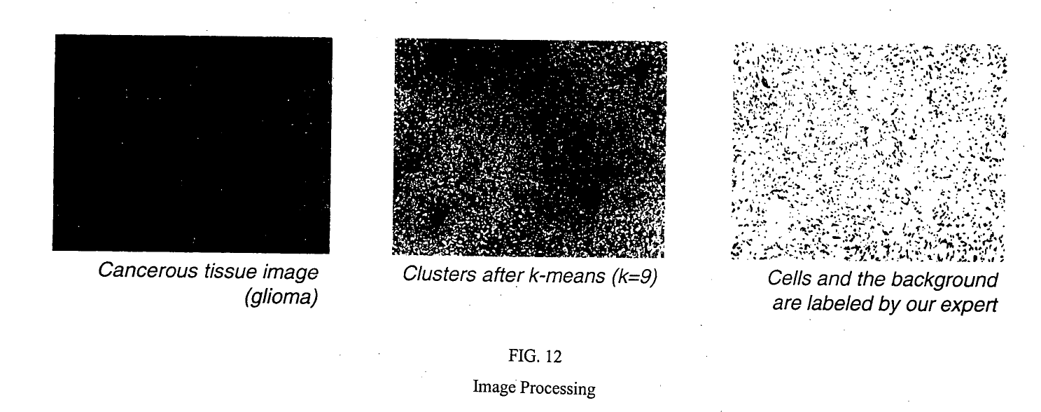

FIG. 12 depicts image processing of cancerous tissue showing a cancerous glioma tissue image (FIG. 12(a)), clusters resulting from application of a K-means algorithm with K=9 (FIG. 12(b)), and cells and the background as labeled by a pathology expert, in accordance with embodiments of the present invention.

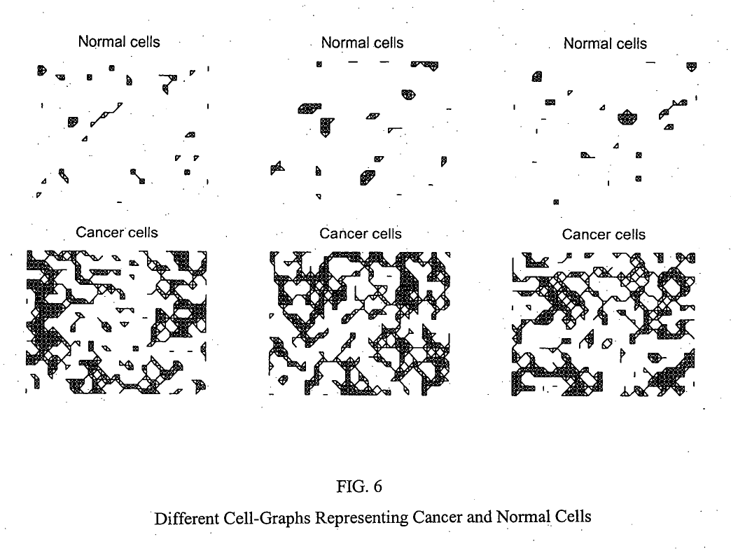

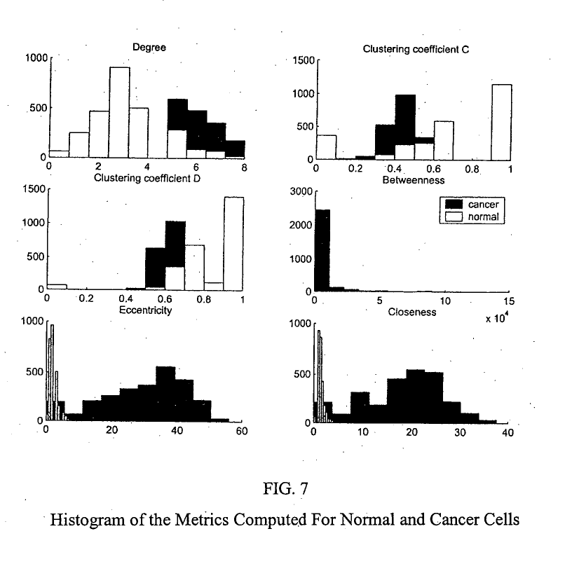

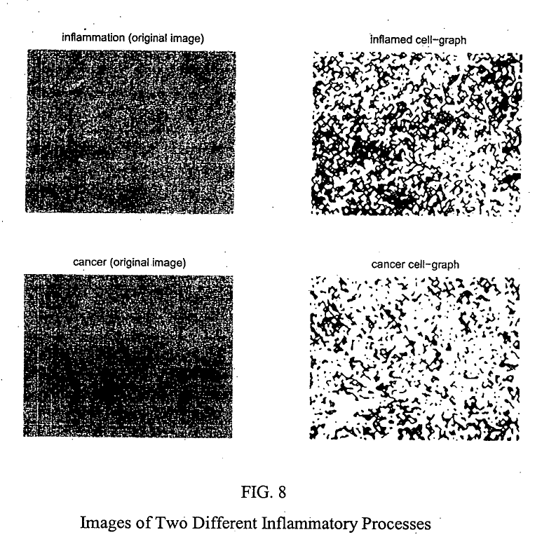

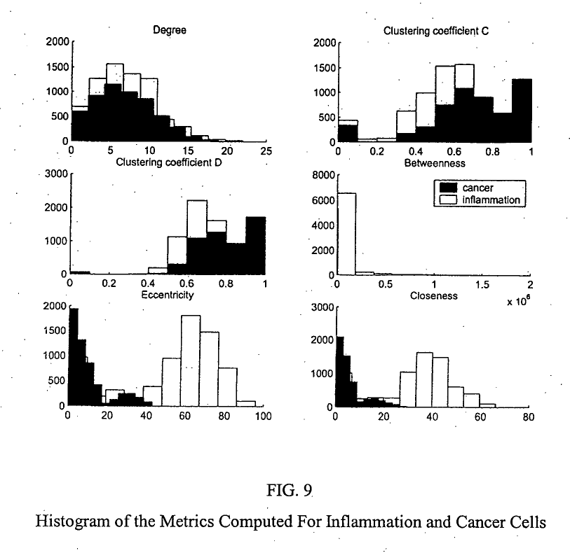

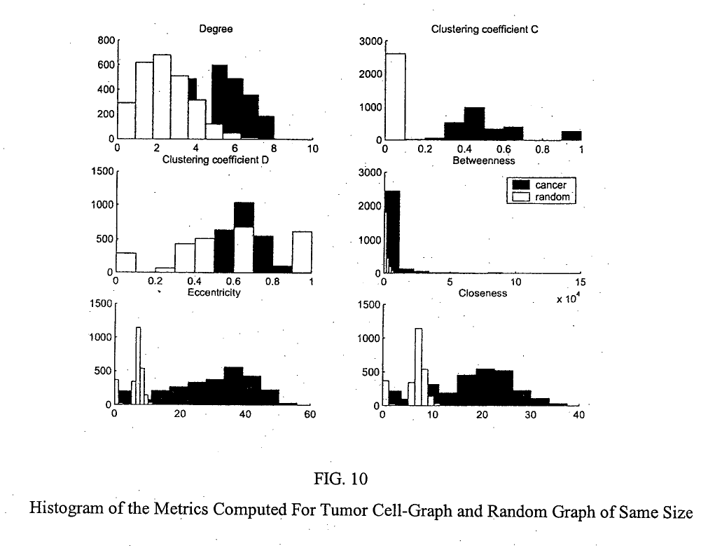

Next, the cell-graphs extracted from the cancerous tissues are compared to the cell-graphs of three different types of structures, namely the cell-graphs of normal tissue (FIGS. 6-7), the cell-graphs of inflamed tissue (FIGS. 8-9), and randomly generated cell-graphs (FIG. 10). These comparisons will demonstrate that the cell-graphs of cancerous tissues are different than those of the three different types of structures, from which it is concluded that the cell-graph structure of glioma differs from the cell-graph structure of other biological phenomenon.

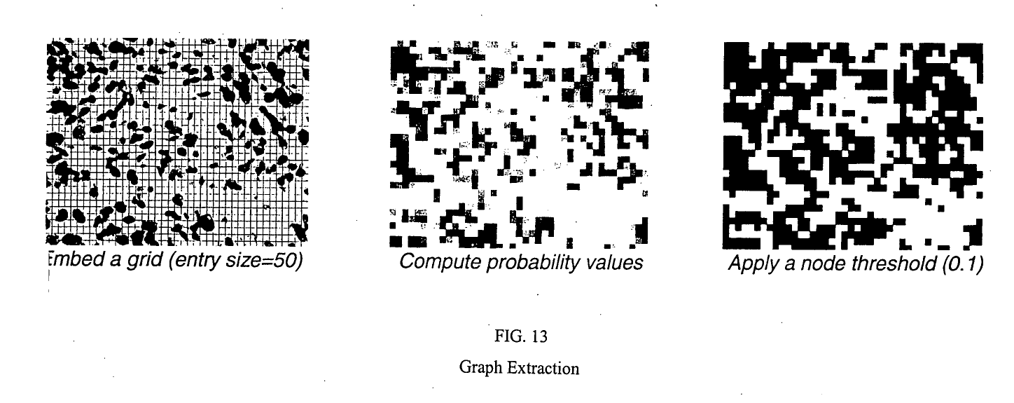

FIG. 6 depicts cell-graphs representing cancer cells from glioma tumor tissue and normal cells, in accordance with embodiments of the present invention. FIG. 13 illustrates a comparison between normal (healthy) tissue and cancer tissue (glioma), in accordance with embodiments of the present invention. In FIG. 6, the sparsity (i.e., density) of the graphs show that the tumor and normal tissues have completely different graphs, which is validated by FIG. 7 depicting data histograms of metrics computed for the cell-graphs representing cancer and normal cells in FIG. 6, in accordance with embodiments of the present invention. The histograms in FIG. 7 are based on five different tissue images of both cancer tissue and normal tissue. The histograms in FIG. 7 are for the metrics of degree, clustering coefficient C, clustering coefficient D, betweenness, eccentricity, and closeness. The difference in the histograms for the cancer and normal cells for each metric provides statistical validation that normal and cancer cells can be distinguished by using these metrics.

FIG. 8 depicts images and cell-graphs representing cancer cells from tumor tissue (upper two sub-figures) and inflammation cells (lower two sub-figures), in accordance with embodiments of the present invention. FIG. 9 depicts data histograms of metrics computed for the image and cell-graphs representing cancer and inflammation cells in FIG. 8, in accordance with embodiments of the present invention. The histograms in FIG. 9 are for the metrics of degree, clustering coefficient C, clustering coefficient D, betweenness, eccentricity, and closeness. FIG. 9 shows that the metrics for the cancerous and inflamed tissues differ the least for the of the indicated metrics. Thus, inflamed tissue and cancerous tissue can be distinguished based on, at least, their respective metrics.

The histograms in FIG. 9 show that it is not as easy as with the histograms of FIG. 7 to distinguish the cancer and inflammation cells. Accordingly, a classifier algorithm was run for cancer and inflammation cells, using a multilayer perceptron with 5 hidden units. Table 1 infra shows its average accuracy results of more than 75 per cent on training and testing sets, which indicates that the classification is based on the metric values. If it were random, the accuracy results would be approximately 50 percent for two classes classification. Therefore, the histograms of FIG. 9, combined with the accuracy results in Table 1, show that the graph structure of glioma is different statistically from the graph structure of inflamed tissue.

TABLE 1
Accuracy values of training and test sets in
classifying inflammation and tumor cells.

| | Average | Standard Deviation |
|---|---|---|
| Training Set | 91.23 | 0.08 |
| Test set | 76.83 | 0.10 |

Random graphs of the same size as the cancer subgraph were generated and the aforementioned metrics were computed on them as depicted in FIG. 10. In particular, FIG. 10 depicts data histograms of metrics computed for the cell-graphs representing cancer cells and for randomly generated cell-graphs, in accordance with embodiments of the present invention. The histograms in FIG. 10 are for the metrics of degree, clustering coefficient C, clustering coefficient D, betweenness, eccentricity, and closeness. Note that the clustering coefficient C is markedly smaller for the cancer graphs than for the random graphs, and the histograms in FIG. 10 show that a tumor cell-graph is different than the random graph.

A classification algorithm was run to distinguish the cancer and normal cell-graphs as well as the random graphs. Using a multilayer perceptron with 5 hidden units, the accuracy values on the training and test sets (for the three classes of normal, cancer, and random) are given in Table 2. From Table 2, it is concluded that the types of nodes can be determined automatically with approximately 95% accuracy.

TABLE 2
Accuracy values on the training and test sets
for classes: normal, cancer, and random.

| | Average | Standard Deviation |
|---|---|---|
| Training Set | 94.98 | 0.05 |
| Test set | 94.52 | 0.08 |

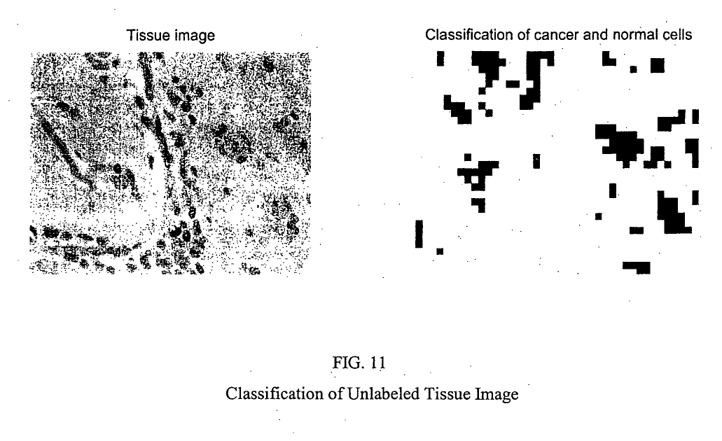

FIG. 11 depicts an image and graph of tissue containing both cancer and normal cells, and a graph classifying cancer and normal cells within the image, in accordance with embodiments of the present invention. The algorithm of the present invention was tested on the images of FIG. 11. These images are not used in training of either K-means algorithm or multilayer perceptrons. In FIG. 11, black regions indicate normal cells, whereas the lighter regions show cancer cells.

In summary, the present invention presents a novel approach for mathematical modeling of diffuse gliomas based on graph theory. The present invention advances the current computational and mathematical modeling approaches by scaling up the cell-graphs with large number of vertices. The graph theoretical model is scalable and used by a machine learning algorithm which can distinguish: (i) gliomas from surrounding normal tissue; and (ii) gliomas from inflammation. The experimental results described herein are based on real data and validate the present invention.

While particular embodiments of the present invention have been described herein for purposes of illustration, many modifications and changes will become apparent to those skilled in the art. Accordingly, the appended claims are intended to encompass all such modifications and changes as fall within the true spirit and scope of this invention.

1. A method for tissue modeling using at least one tissue image derived from clinical tissue, said at least one tissue image having cells therein, said method comprising for each tissue image of the at least one tissue image wherein each tissue image is denoted as a sample tissue image:
clustering data derived from the sample tissue image to generate cluster vectors, each cluster vector representing of portion of the tissue image;
generating cell information, comprising assigning a cell class or a background class to each of the cluster vectors;
generating a cell-graph for the sample tissue image from using the generated cell information, said cell-graph comprising nodes and edges, said edges connecting some of the cell nodes together based on a connectivity criterion; and
computing at least one metric from the generated cell-graph.

2. The method of claim 1, said clinical tissue having been surgically removed from at least one patient.

3. The method of claim 1, said at least one metric being selected from the group consisting of degree, at least one clustering coefficient, closeness, betweenness, eccentricity, and combinations thereof.

4. The method of claim 1, said method further comprising for the sample tissue image:
classifying the sample tissue image to determine whether or not the cell nodes of the sample tissue image represent cancer cells, by utilizing the computed at least one metric.

5. The method of claim 4, said classifying comprising executing a machine learning algorithm that employs neural networks.

6. The method of claim 1, said at least one tissue image comprising at least one tissue image having cancer cells therein and at least one tissue image having inflammation cells therein, said method further comprising:
generating a first data histogram representing a first metric of the at least one metric for the generated cell-graph of the at least one tissue image having cancer cells therein; and
generating a second data histogram representing a second metric of the at least one metric for the generated cell-graph of the at least one tissue image having inflammation cells therein, said first and second metric being a same metric, and
displaying the first data histogram and the second data histogram together on a single graph to facilitate a visual comparison between the first data histogram and the second data histogram.

7. The method of claim 6, said at least one tissue image comprising at least one tissue image having cancer cells therein being first tissue images, said at least one tissue image having inflammation cells being second tissue images, said method further comprising:
classifying the first tissue images to determine whether or not the cell nodes of the first tissue images represent cancer cells, by utilizing the computed at least one metric for the first tissue images;
classifying the second tissue images to determine whether or not the cell nodes of the second tissue images represent inflammation cells, by utilizing the computed at least one metric for the second tissue images; and
determining an average accuracy of said classifying the first and second tissue images.

8. The method of claim 1, said at least one tissue image comprising at least one tissue image having cancer cells therein and at least one tissue image having normal cells therein, said normal cells representing healthy tissue, said method further comprising:
generating a first data histogram representing a first metric of the at least one metric for the generated cell-graph of the at least one tissue image having cancer cells therein; and
generating a second data histogram representing a second metric of the at least one metric for the generated cell-graph of the at least one tissue image having normal cells therein, said first and second metric being a same metric, and
displaying the first data histogram and the second data histogram together on a single graph to facilitate a visual comparison between the first data histogram and the second data histogram.

9. The method of claim 8, said at least one tissue image comprising at least one tissue image having cancer cells therein being first tissue images, said at least one tissue image having normal cells being second tissue images, said method further comprising:
classifying the first tissue images to determine whether or not the cell nodes of the first tissue images represent cancer cells, by utilizing the computed at least one metric for the first tissue images;
classifying the second tissue images to determine whether or not the cell nodes of the second tissue images represent normal cells, by utilizing the computed at least one metric for the second tissue images; and
determining an average accuracy of said classifying the first and second tissue images.

10. An apparatus for implementing the method of claim 1, said apparatus comprising:
means for clustering the data derived from the sample tissue image;
means for generating the cell information;
means for generating the cell-graph for the sample tissue image; and
means for computing the at least one metric.
